(19) United States
(12) Patent Application Publication
(10) Pub. No.: US 2023/0191006 A1
(43) Pub. Date: Jun. 22, 2023

(54) BLOOD BAG SYSTEM, CENTRIFUGAL SEPARATION SYSTEM, AND CLAMP

(71) Applicant: TERUMO KABUSHIKI KAISHA, Tokyo (JP)

(72) Inventors: Masatsugu IGARASHI, Kanagawa (JP); Tadashi IMAI, Shizuoka (JP); Yuusuke YAMAZAKI, Tokyo (JP)

(21) Appl. No.: 18/170,430

(22) Filed: Feb. 16, 2023

Related U.S. Application Data
(63) Continuation of application No. PCT/JP2021/029887, filed on Aug. 16, 2021.

(30) Foreign Application Priority Data
Aug. 18, 2020 (JP) 2020-137881

(51) Int. Cl. A61M 1/02; A61M 1/36; A61M 39/28

(52) U.S. Cl. CPC A61M 1/0218; A61M 1/3693; A61M 39/28

(57) ABSTRACT

A centrifugal separation system includes a blood bag system and a centrifugal separation and transferring device. The blood bag system is equipped with a single clamp that is mounted over a second tube and a third tube. In a first blocking portion of a first insertion hole of the clamp, a portion thereof except for a portion connected with a first opening portion is closed by a main body. On the other hand, a second insertion hole of the clamp includes a tube detaching portion, that is connected with a second blocking portion and that enables the third tube to be detached from the main body.

### CROSS-REFERENCE TO RELATED APPLICATIONS

The present disclosure is a continuation of and claims benefit to PCT/JP2021/029887 filed on Aug. 16, 2021, entitled “BLOOD BAG SYSTEM, CENTRIFUGAL SEPARATION SYSTEM, AND CLAMP” which claims priority to Japanese Patent Application No. 2020-137881A filed on Aug. 18, 2020. The entire disclosures of the applications listed above are hereby incorporated herein by reference, in their entireties, for all that they teach and for all purposes.

### BACKGROUND

The present disclosure relates to a blood bag system having a plurality of tubes and a clamp for opening and closing flow paths of the tubes, a centrifugal separation system to which the blood bag system is applied, and a clamp.

In Japanese Patent Application No. 2016-106012 A, a blood bag system is disclosed, that is equipped with a first bag (a parent bag) for accommodating blood, a second bag (a child bag) for accommodating blood components obtained by centrifugally separating the blood inside the first bag, and a third bag (a chemical solution bag) accommodating an additive solution. The blood bag system includes a first tube connected with the first bag, a second tube connected with the second bag, and a third tube connected with the third bag, and the first to third tubes are connected with each other by way of a branching portion (a Y-type connector).

By being set in a centrifugal separation and transferring device by a user such as a medical worker or the like, the blood bag system constitutes a centrifugal separation system in which the blood is subjected to centrifugal separation to generate blood components, and a liquid containing the blood components is transferred between the first to third bags. In the centrifugal separation system, after a blood component has been transferred from the first bag to the second bag, a flow path of the second tube is blocked by the user by a first clamp, whereas a flow path of the third tube is blocked by a second clamp. In this state, the user takes out the blood bag system from the centrifugal separation and transferring device, suspends the blood bag system on a suspension platform, and further separates the second clamp from the third tube, thereby transferring the additive solution from the third bag into the first bag.

### BRIEF SUMMARY

Incidentally, in such a centrifugal separation system (e.g., a blood bag system), with the first clamp and the second clamp being provided as separate components, it is likely that the user will consume time and effort in order to respectively block the flow path of the second tube and the flow path of the third tube. Further, in an operation of unfastening the second clamp, there is a concern that the user may accidentally unfasten the first clamp. In other words, there is a concern that the wrong tube may be opened by mistake.

The present disclosure has been devised taking into consideration the aforementioned problems, and has at least one object of providing a blood bag system, a centrifugal separation system, and a clamp, that are capable of efficiently blocking the flow paths of two tubes by a single clamp, and preventing the flow path of the tube from being opened by mistake.

A first aspect of the present disclosure is characterized by a blood bag system, comprising a first bag configured to accommodate blood, a second bag configured to accommodate a blood component obtained by subjecting the blood inside the first bag to centrifugal separation, a third bag configured to accommodate an additive solution, a first tube connected to the first bag, a second tube connected to the second bag, and a third tube connected to the third bag, the first tube, the second tube, and the third tube being connected via a branching portion, the blood bag system further comprising a single clamp mounted over the second tube and the third tube, wherein the clamp includes a main body, a first insertion hole that penetrates, or passes, through the main body and in which the second tube is arranged, and a second insertion hole that penetrates, or passes, through the main body and in which the third tube is arranged, the first insertion hole includes a first opening portion configured to place a flow path of the second tube in an open state, and a first blocking portion connected with the first opening portion and configured to place the flow path of the second tube in a blocked state, and in the first blocking portion, a portion thereof except for a portion connected with the first opening portion is closed by the main body, and the second insertion hole includes a second opening portion configured to place a flow path of the third tube in an open state, a second blocking portion connected with the second opening portion and configured to place the flow path of the third tube in a blocked state, and a tube detaching portion connected with the second blocking portion and configured to enable the third tube to be detached from the main body.

Further, a second aspect of the present disclosure is characterized by a centrifugal separation system, comprising: a blood bag system including a first bag configured to accommodate blood, a second bag configured to accommodate a blood component obtained by subjecting the blood inside the first bag to centrifugal separation, a third bag configured to accommodate an additive solution, a first tube connected to the first bag, a second tube connected to the second bag, and a third tube connected to the third bag, the first tube, the second tube, and the third tube being connected via a branching portion; and a centrifugal separation and transferring device in which the blood bag system is set, the centrifugal separation and transferring device being configured to subject the blood in the first bag to centrifugal separation and transfer the blood component into the second bag, wherein the blood bag system includes a single clamp mounted over the second tube and the third tube, the clamp includes a main body, a first insertion hole that penetrates, or passes, through the main body and in which the second tube is arranged, and a second insertion hole that penetrates, or passes, through the main body and in which the third tube is arranged, the first insertion hole includes a first opening portion configured to place a flow path of the second tube in an open state, and a first blocking portion connected with the first opening portion and configured to place the flow path of the second tube in a blocked state, and in the first blocking portion, a portion thereof except for a portion connected with the first opening portion is closed by the main body, the second insertion hole includes a second opening portion configured to place a flow path of the third tube in an open state, a second blocking portion connected with the second opening portion and configured to place the flow path of the third tube in a blocked state, and a tube detaching portion connected with the second blocking portion and configured to enable the third tube to be detached from the main body, and the centrifugal separation and transferring device includes a holder configured to retain the branching portion and the clamp.

Furthermore, a third aspect of the present disclosure is characterized by a clamp mounted over two tubes, the clamp comprising a main body, a first insertion hole that penetrates, or passes, through the main body and in which one tube of the two tubes is arranged, and a second insertion hole that penetrates, or passes, through the main body and in which another tube of the two tubes is arranged, wherein the first insertion hole includes a first opening portion configured to place a flow path of the one tube in an open state, and a first blocking portion connected with the first opening portion and configured to place the flow path of the one tube in a blocked state, and in the first blocking portion, a portion thereof except for a portion connected with the first opening portion is closed by the main body, and the second insertion hole includes a second opening portion configured to place a flow path of the another tube in an open state, a second blocking portion connected with the second opening portion and configured to place the flow path of the another tube in a blocked state, and a tube detaching portion connected with the second blocking portion and configured to enable the another tube to be detached from the main body.

With the blood bag system, the centrifugal separation system, and the clamp described above, it is possible to efficiently block the flow paths of the two tubes by a single clamp, and prevent the flow path of the tube from being opened by mistake.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is an explanatory diagram showing an overall configuration of a blood bag system and a centrifugal separation system according to a first embodiment of the present disclosure;

FIG. 2 is a plan view showing a unit setting area of a centrifugal separation and transferring device in accordance with embodiments of the present disclosure;

FIG. 3 is a perspective view showing a state in which a clamp of the blood bag system is set in a holder of the centrifugal separation and transferring device in accordance with embodiments of the present disclosure;

FIG. 4 is a front view of a clamp according to the first embodiment of the present disclosure;

FIG. 5A is a perspective view of a holder in accordance with embodiments of the present disclosure;

FIG. 5B is a first explanatory diagram showing a sliding operation of the clamp that is retained by the holder in accordance with embodiments of the present disclosure;

FIG. 5C is a second explanatory diagram showing a sliding operation of the clamp that is retained by the holder in accordance with embodiments of the present disclosure;

FIG. 6 is a flowchart showing a flow of operations when the centrifugal separation system performs centrifugal separation in accordance with embodiments of the present disclosure;

FIG. 7A is a perspective view showing an operation of the clamp in a clamp blocking step in accordance with embodiments of the present disclosure;

FIG. 7B is a perspective view showing operations of the clamp and the third tube in an additive solution injection step in accordance with embodiments of the present disclosure;

FIG. 8 is a front view for describing a clamp according to another exemplary configuration of the present disclosure;

FIG. 9 is a perspective view showing a state in which a clamp of a blood bag system according to a second embodiment of the present disclosure is set in a holder of the centrifugal separation and transferring device;

FIG. 10 is a front view for describing a clamp according to the second embodiment of the present disclosure;

FIG. 11A is a perspective view showing an operation of the clamp in a clamp blocking step in accordance with embodiments of the present disclosure; and

FIG. 11B is a perspective view showing operations of the clamp and the third tube in an additive solution injection step in accordance with embodiments of the present disclosure.

### DETAILED DESCRIPTION

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The ensuing description provides embodiments only, and is not intended to limit the scope, applicability, or configuration of the claims. Rather, the ensuing description will provide those skilled in the art with an enabling description for implementing the described embodiments. It being understood that various changes may be made in the function and arrangement of elements without departing from the spirit and scope of the appended claims.

Various aspects of the present disclosure will be described herein with reference to drawings that may be schematic illustrations of idealized configurations.

Embodiments of the present disclosure will be presented and described in detail below with reference to the accompanying drawings.

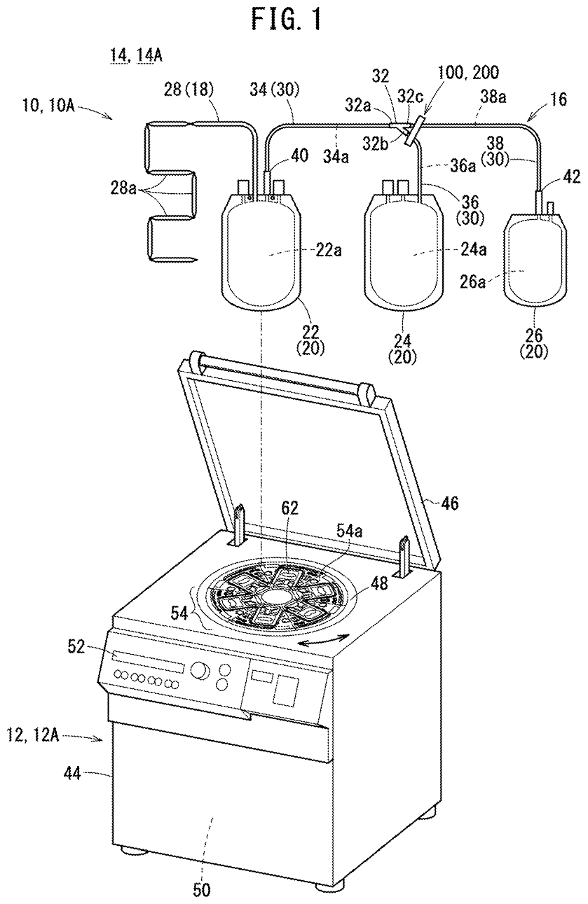

A blood bag system 10 according to a first embodiment of the present disclosure is set in a centrifugal separation and transferring device 12 as shown in FIG. 1, by a user such as a medical worker, technician, and/or the like. The centrifugal separation and transferring device 12 subjects the blood of the blood bag system 10 to centrifugal separation to thereby generate a plurality of types of blood components, and transfers and stores the generated blood components in appropriate bags 20. Hereinafter, a configuration including the blood bag system 10 and the centrifugal separation and transferring device 12 will be referred to as a centrifugal separation system 14.

The blood bag system 10 includes a non-illustrated preprocessing unit used for blood collection prior to centrifugal separation, a separation processing unit 16 that generates the blood components by way of centrifugal separation and separately stores the blood components, and a relay tube 18 connecting the preprocessing unit and the separation processing unit 16 together. Prior to centrifugal separation, in a state in which the relay tube 18 connected to the preprocessing unit is disconnected as shown in FIG. 1, the separation processing unit 16 is set in the centrifugal separation and transferring device 12.

The preprocessing unit of the blood bag system 10 collects whole blood from a blood donor, and further, removes blood components such as leukocytes from the whole blood. Therefore, the preprocessing unit includes a blood collection needle, a blood collection bag, an initial flow blood collection bag, and a filter (for example, a leukocyte removal filter) and the like, none of which are shown, and is constituted by connecting the respective members by a plurality of tubes 30. More specifically, the preprocessing unit stores, in the initial flow blood collection bag, initial blood from among the whole blood of the donor collected through the blood collection needle, and thereafter, stores the remaining blood in the blood collection bag. Furthermore, by the whole blood stored in the blood collection bag passing through the filter, the preprocessing unit removes the leukocytes in the filter. Then, after the leukocytes have been removed, the blood is transferred into the separation processing unit 16 that is connected to the filter via the relay tube 18.

The separation processing unit 16 of the blood bag system 10 includes a plurality of bags 20 (e.g., a blood bag 22, a PPP bag 24, and a chemical solution bag 26), and is constituted by connecting the respective bags 20 by the plurality of tubes 30.

The blood bag 22 is directly connected to the relay tube 18 that is connected with the preprocessing unit, in an initial state of the blood bag system 10 before use. The blood bag 22 is a first bag having a first storage space 22a for storing blood from which leukocytes have been removed by passing through the filter at the time of blood collection. The blood bag 22 is set in the centrifugal separation and transferring device 12, and is applied with a centrifugal force by operation of the centrifugal separation and transferring device 12. Consequently, the removed blood in the blood bag 22 is subjected to centrifugal separation into blood components such as platelet poor plasma (PPP), concentrated red blood cells (RBC), and the like having different specific gravities. In addition, after the centrifugal separation, by transferring the PPP under operation of the centrifugal separation and transferring device 12, the blood bag 22 stores only the remaining RBC.

The PPP bag 24 is a second bag (e.g., a plasma bag) that stores, in a second storage space 24a, the PPP that is supplied thereto from the blood bag 22. On the other hand, the chemical solution bag 26 is a third bag that accommodates beforehand, in a third storage space 26a, a red blood cell preservative solution (hereinafter, referred to as an additive solution) such as a Mannitol-Adenine-Phosphate (MAP) solution, a Saline Adenine Glucose Mannitol (SAGM) solution, OPTISOL solution, and/or the like.

Further, prior to the separation processing unit 16 being set in the centrifugal separation and transferring device 12, the relay tube 18 is sealed at predetermined intervals to thereby form tube segments 28.

The plurality of tubes 30 of the separation processing unit 16 are branched in multiple directions via a branching connector 32 (Y-type connector: branching portion). In greater detail, the plurality of tubes 30 include a first tube 34 that connects the blood bag 22 and the branching connector 32, a second tube 36 that connects the PPP bag 24 and the branching connector 32, and a third tube 38 that connects the chemical solution bag 26 and the branching connector 32. The first tube 34 includes a first flow path 34a, the second tube 36 includes a second flow path 36a, and the third tube 38 includes a third flow path 38a.

Further, the branching connector 32 includes a first port 32a to which the first tube 34 is connected, a second port 32b to which the second tube 36 is connected, and a third port 32c to which the third tube 38 is connected, and the first to third ports 32a to 32c are integrally molded. The first to third ports 32a to 32c communicate with each other by a non-illustrated internal flow path, whereby the respective flow paths of the first to third tubes 34, 36, and 38 are placed in communication with each other.

At an end of the first tube 34 on the side of the blood bag 22, there is provided a sealing member 40 that is capable of being broken, and includes an internal flow path that opens accompanying breaking of the tube by the user. Similarly, a sealing member 42 that is capable of being broken is provided at an end of the third tube 38 on the side of the chemical solution bag 26. The sealing members 40 and 42 block the first flow path 34a and the third flow path 38a respectively until the breaking operation is performed, and serve to prevent the blood inside the blood bag 22 and the additive solution inside the chemical solution bag 26 from being transferred into the other bags 20.

Further, the blood bag system 10 is equipped with a single clamp 100 that is mounted over the second tube 36 and the third tube 38 at positions in the vicinity of the branching connector 32. The clamp 100 is configured to be capable of switching the second flow path 36a of the second tube 36 and the third flow path 38a of the third tube 38 from an open state to a blocked state. A description will be given in detail later concerning the specific configuration of the clamp 100.

On the other hand, the centrifugal separation and transferring device 12 in which the separation processing unit 16 of the blood bag system 10 is set is equipped with a box-shaped base body 44, a lid 46 that is capable of opening and closing an upper surface of the base body 44, and a centrifugal drum 48 provided on the base body 44. Further, a non-illustrated motor for causing the centrifugal drum 48 to rotate, a control unit 50 for controlling operations of the centrifugal separation and transferring device 12, and an operation display unit 52 in order for the user to confirm and carry out operations are provided on the base body 44 of the centrifugal separation and transferring device 12.

The centrifugal drum 48 includes a plurality of (six) unit setting areas 54 in which the separation processing unit 16 can be set. The height of one of the unit setting areas 54 is greater than a longitudinal length of the bags 20, and is set within a range of 60 degrees with respect to the center of rotation of the centrifugal drum 48. Stated another way, the centrifugal drum 48, which may be configured as an annular structural member, is constituted by arranging the six unit setting areas 54 alongside one another without gaps therebetween along a circumferential direction.

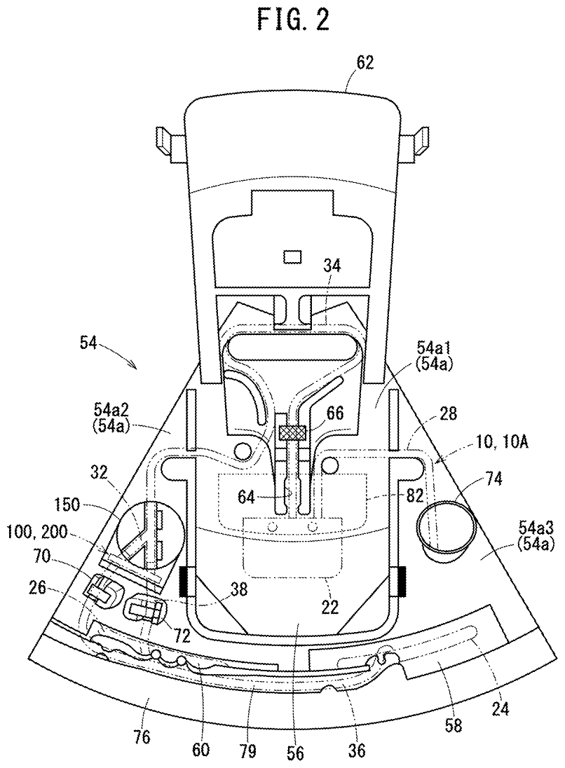

As shown in FIG. 2, the unit setting area 54 is equipped, at positions on an outer side in a radial direction of the centrifugal drum 48, with a blood bag pocket 56 in which the blood bag 22 is accommodated, a PPP bag pocket 58 in which the PPP bag 24 is accommodated, and a chemical solution bag pocket 60 in which the chemical solution bag 26 is accommodated.

The blood bag pocket 56 is provided in a central portion in the circumferential direction of the unit setting area 54, and has a volume that is greater than that of the PPP bag pocket 58 and the chemical solution bag pocket 60. The PPP bag pocket 58 and the chemical solution bag pocket 60 are disposed alongside one another in the circumferential direction, on a more outer side in the radial direction than the blood bag pocket 56.

Further, an upper surface 54a of the unit setting area 54 is configured such that the plurality of tubes 30 of the blood bag system 10 are arranged and retained thereon. On the upper surface 54a, the first tube 34 and one portion of the tube segments 28 are arranged in a central region 54a1 on a more inner side in the radial direction than the blood bag pocket 56. Further, in the central region 54a1, a lid 62 is provided that opens and closes an opening of the blood bag pocket 56. Furthermore, a portion of a breaking member 64 that serves to break the sealing member 40, and a sensor 66 that detects a state of the blood flowing through the first tube 34 are disposed at a position where the first tube 34 that is covered by the lid 62 is arranged.

In a left region 54a2 of the upper surface 54a, one portion of the first tube 34 after having passed through the central region 54a1, the branching connector 32, the second tube 36, and one portion of the third tube 38 are arranged. In the left region 54a2, a holder 150 for retaining the branching connector 32, a PPP clamp 70 for opening and closing the second flow path 36a of the second tube 36, and a chemical solution clamp 72 for opening and closing the third flow path 38a of the third tube 38 are provided.

In a right region 54a3 of the upper surface 54a, a segment pocket 74 is provided in which a plurality of hermetically sealed tubes 28a (see, e.g., FIG. 1) of the tube segments 28 are accommodated.

Furthermore, a tube retaining member 76 that projects upwardly from the upper surface 54a is provided on a more outer side in the radial direction than the PPP bag pocket 58 and the chemical solution bag pocket 60 in the unit setting area 54. The tube retaining member 76 includes a guide groove portion 79 for allowing the second tube 36 to be arranged therein.

Further, the unit setting area 54 is equipped with a slider 82 that presses on the blood bag 22 following completion of centrifugal separation, on an inner side in the radial direction of the blood bag pocket 56. The slider 82 advances and retracts along the radial direction of the centrifugal drum 48 under the control of the control unit 50 (see, e.g., FIG. 1).

In the above-described unit setting area 54, the user places, in the blood bag pocket 56, the blood bag 22 in which the removed blood is stored, places the empty PPP bag 24 in the PPP bag pocket 58, and places, in the chemical solution bag pocket 60, the chemical solution bag 26 in which the chemical solution is stored, respectively. In addition, in the unit setting area 54, the removed blood of the blood bag 22 is subjected to centrifugal separation by rotation of the centrifugal drum 48, and after centrifugal separation is completed, the slider 82 is advanced and presses on the blood bag 22. Consequently, the PPP, which is generated by centrifugal separation in the blood bag 22, is transferred into the PPP bag 24, whereas the RBC remains in the blood bag 22 after the PPP has been transferred. Following transfer of the PPP, the blood bag system 10 is taken out from the centrifugal separation and transferring device 12 by the user.

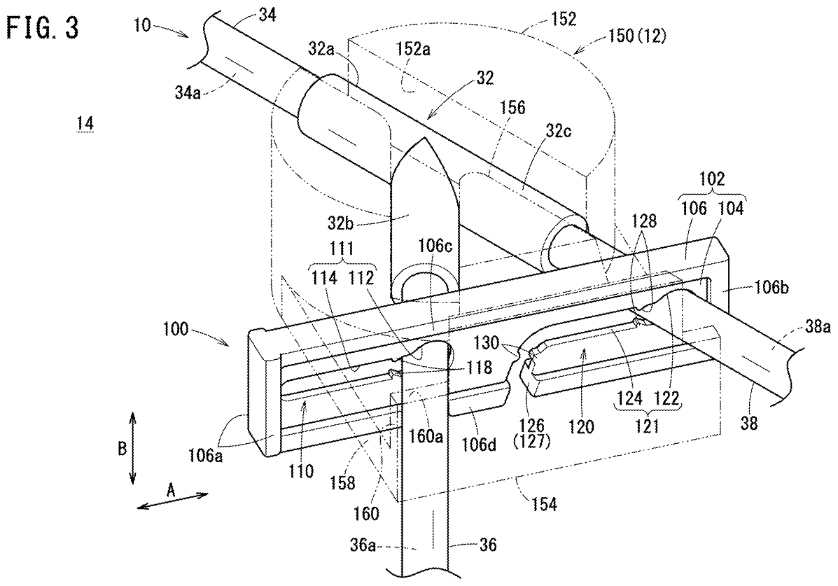

Next, a description is provided with reference to FIG. 3 concerning the clamp 100 of the blood bag system 10 according to the present embodiment.

As described above, the clamp 100 is mounted over the second tube 36 and the third tube 38 of the blood bag system 10, and is configured to enable the second flow path 36a and the third flow path 38a to be switched from an open state to a blocked state. Furthermore, the clamp 100 is configured so as to enable the third tube 38 to be detached from the clamp 100. By being detached in this manner, the third flow path 38a of the third tube 38 is opened.

More specifically, the clamp 100 comprises a plate-shaped main body 102 constituted by a resin material or a metal material. In addition, the clamp 100 includes a first insertion hole 110 that penetrates, or passes, through the main body 102 and in which the second tube 36 is arranged, and a second insertion hole 120 that penetrates, or passes, through the main body 102 and in which the third tube 38 is arranged.

The main body 102 is configured so as to possess a higher rigidity (a greater elastic modulus and hardness) than the tubes 30. The main body 102 includes a base 104 having a horizontally elongate rectangular shape, and an outer peripheral frame 106 that encircles the outer periphery of the base 104. The main body 102 is formed in a rectangular shape when viewed from the front in a thickness direction. The base 104 and the outer peripheral frame 106 are integrally molded from the same material.

When the blood bag system 10 is used, the base 104 constitutes a portion that is placed in direct contact with the second tube 36 and the third tube 38, and switches between the open state and the blocked state of the second flow path 36a and the third flow path 38a. The base 104 is formed in a flat plate-like shape having a first surface and a second surface, and the first insertion hole 110 and the second insertion hole 120 penetrate, or pass, through the first surface and the second surface in the thickness direction.

The base 104 has an appropriate thickness in order to constitute an edge portion that is capable of being placed in surface contact with the outer circumferential surface of the second tube 36 and the outer circumferential surface of the third tube 38. The thickness of the base 104 is not particularly limited, but may be set, for example, on the order of 0.5 mm to 2 mm.

In order to reinforce the rigidity of the base 104, the outer peripheral frame 106 is formed so as to possess a greater thickness than the base 104. A pair of flange portions 106a are disposed on both sides in the thickness direction of one end of the outer peripheral frame 106 in the longitudinal direction (the direction of the arrow A). The pair of flange portions 106a project in the thickness direction of the main body 102 more so than other portions (a short side 106b on the other end side in the longitudinal direction, and a pair of long sides 106c and 106d) of the outer peripheral frame 106. The flange portions 106a are provided in order to make it easier for the operator to contact the clamp 100 with the fingers when pushing the clamp 100.

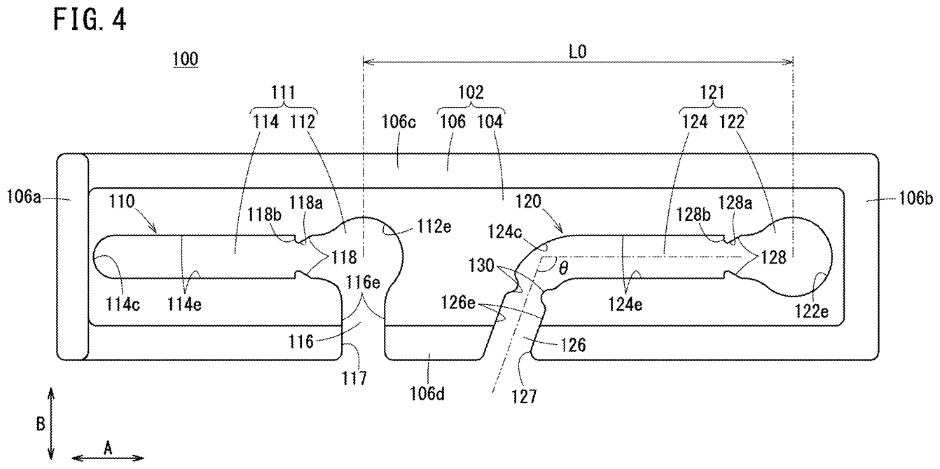

As shown in FIGS. 3 and 4, the first insertion hole 110 and the second insertion hole 120 are formed by inner edge portions included on an inner side of the main body 102. The first insertion hole 110 and the second insertion hole 120 are disposed side by side in a central portion in a lateral direction (vertical direction: the direction of the arrow B) of the main body 102. A main portion of the first insertion hole 110 (a first space 111 for opening and closing the second flow path 36a of the second tube 36) and a main portion of the second insertion hole 120 (a second space 121 for opening and closing the third flow path 38a of the third tube 38) both extend along the longitudinal direction (horizontal direction) of the main body 102. More specifically, the first space 111 and the second space 121 are arranged in a row alongside one another, and extend in the same direction.

The first space 111 includes a first opening portion 112 that places the second flow path 36a of the second tube 36 in an open state, and a first blocking portion 114 that is connected with the first opening portion 112 and places the second flow path 36a of the second tube 36 in a blocked state. Further, the first insertion hole 110 includes a tube introducing portion 116 that is connected with the first opening portion 112 and is capable of introducing the second tube 36 into the first opening portion 112.

The first opening portion 112 is disposed in the vicinity of the center in the longitudinal direction of the main body 102. The first opening portion 112 is formed as a substantially perfect circular shaped space by a first arcuate edge portion 112e of the main body 102, and has a diameter greater than or equal to the outer diameter of the second tube 36. For example, the diameter of the first opening portion 112 is set on the order of 1.0 to 1.5 times the outer diameter of the second tube 36.

The first blocking portion 114 extends from the first opening portion 112 toward one end side in the longitudinal direction where the flange portions 106a of the main body 102 are located. The first blocking portion 114 is formed as a horizontal elongate space by a pair of first straight edge portions 114e extending in the longitudinal direction of the base 104, and with a width that is narrower than the outer diameter of the second tube 36. When the second tube 36 is moved into the first blocking portion 114, the pair of first straight edge portions 114e act to block the second flow path 36a of the second tube 36 by pressing inwardly on the outer circumferential surface of the second tube 36. In order for the second tube 36 to be easily inserted from the first opening portion 112 into the first blocking portion 114, the first insertion hole 110 may have a configuration in which the width between the pair of first straight edge portions 114e is narrowed in a tapered shape so that the edge portions come into closer proximity from the first opening portion 112 toward the inner back side of the first blocking portion 114.

One end side in the longitudinal direction of the first blocking portion 114 extends to the vicinity of the flange portions 106a, and is closed by a semicircular-shaped first blocking edge portion 114c. More specifically, in the first blocking portion 114, a portion thereof except for a portion connected with the first opening portion 112 is surrounded by the main body 102. Accordingly, the first insertion hole 110 is configured in a manner so that the second tube 36 is not detached from the first blocking portion 114 directly toward the exterior of the main body 102.

Further, the clamp 100 includes a pair of first protrusions 118 on the first blocking portion 114 that project toward an inner side of the first insertion hole 110. The pair of first protrusions 118 are disposed at positions in the vicinity of the first opening portion 112. Moreover, the pair of first protrusions 118 may be provided at a boundary between the first opening portion 112 and the first blocking portion 114.

The pair of first protrusions 118 are connected respectively to one and the other of the pair of first straight edge portions 114e. The pair of first protrusions 118 project in directions to approach toward each other, thereby narrowing the width of the first blocking portion 114. Moreover, a first protrusion 118 may be provided on only one of the pair of first straight edge portions 114e.

More specifically, each of the first protrusions 118 is formed in a substantially right triangular shape having an inclined side 118a that is inclined with respect to the longitudinal direction of the first blocking portion 114, and a perpendicular side 118b that is connected to the inclined side 118a via a top rounded corner portion and is perpendicular to the longitudinal direction of the first blocking portion 114. In addition, the inclined sides 118a are provided so as to face the side of the first opening portion 112, and the perpendicular sides 118b are provided so as to face the first blocking edge portion 114c. The pair of first protrusions 118 formed in this manner allow the second tube 36 to be moved from the first opening portion 112 into the first blocking portion 114, while preventing movement of the second tube 36 from the first blocking portion 114 into the first opening portion 112.

The tube introducing portion 116 is disposed in a vertical direction of the first opening portion 112 (the lateral direction of the main body 102), and enables the second tube 36 to be inserted into the first opening portion 112 from the exterior of the main body 102. More specifically, the tube introducing portion 116 is constituted by a pair of first straight edge portions 116e connected with the first arcuate edge portion 112e, and extending in a direction substantially perpendicular to the direction in which the first blocking portion 114 extends. Since the clamp 100 can be easily inserted onto the second tube 36 due to the presence of the tube introducing portion 116, efficiency at the time of manufacturing is improved. It should be noted that the tube introducing portion 116 need not necessarily be provided.

The pair of first straight edge portions 116e form, on the long side 106d, an introduction opening 117 which allows the first insertion hole 110 to communicate with the outer side of the main body 102. The width between the pair of first straight edge portions 116e is formed to be narrower than the diameter of the second tube 36, while being set to be wider than or approximately the same as the width between the pair of first straight edge portions 114e.

On the other hand, the second space 121 includes a second opening portion 122 that places the third flow path 38a of the third tube 38 in an open state, and a second blocking portion 124 that is connected with the second opening portion 122 and places the third flow path 38a of the third tube 38 in a blocked state. Furthermore, the second insertion hole 120 includes a tube detaching portion 126 that is connected with the second blocking portion 124 and which enables the second tube 36 to be detached from the main body 102.

The second opening portion 122 is disposed in the vicinity of the other end in the longitudinal direction of the main body 102. An interval, or distance, LO between a central part of the first opening portion 112 and a central part of the second opening portion 122 is set to a dimension that is one half the length in the longitudinal direction of the main body 102. The second opening portion 122 is formed as a substantially perfect circular shaped space by a second arcuate edge portion 122e of the main body 102, and has a diameter greater than or equal to the outer diameter of the third tube 38. For example, the diameter of the second opening portion 122 is set on the order of 1.0 to 1.5 times the outer diameter of the third tube 38.

The second blocking portion 124 extends from the second opening portion 122 toward a central portion in the longitudinal direction of the main body 102. The second blocking portion 124 is formed as a horizontal elongate space by a pair of second straight edge portions 124e extending in the longitudinal direction of the base 104, and with a width that is narrower than the outer diameter of the third tube 38. In the present embodiment, the width between the pair of second straight edge portions 124e is the same as the width between the pair of first straight edge portions 114e. When the third tube 38 is moved into the second blocking portion 124, the pair of second straight edge portions 124e act to block the third flow path 38a of the third tube 38 by pressing inwardly on the outer circumferential surface of the third tube 38. In order for the third tube 38 to be easily inserted from the second opening portion 122 into the second blocking portion 124, the second insertion hole 120 may have a configuration in which the width between the pair of second straight edge portions 124e is narrowed in a tapered shape so that the edge portions come into closer proximity from the second blocking portion 124 toward the inner back side of the second blocking portion 124.

One end side in the longitudinal direction of the second blocking portion 124 communicates with the tube detaching portion 126 in the vicinity of the center in the longitudinal direction of the main body 102. A pair of arcuate curved portions 124c are formed at one end in the longitudinal direction of the second blocking portion 124, in a smoothly continuous manner from the second straight edge portions 124e to the tube detaching portion 126.

The tube detaching portion 126 extends in a direction that differs from (in a non-parallel direction to) the direction in which the second blocking portion 124 extends. A bending angle theta between the direction of extension of the tube detaching portion 126 and the direction of extension of the second blocking portion 124 is not particularly limited, but may be set within a range of from 20 degrees to 150 degrees, for example. If the bending angle theta were made less than 20 degrees, it would become difficult for the third tube 38 to be taken out from the second blocking portion 124, whereas if the bending angle theta were made greater than 150 degrees, there is a possibility of the second insertion hole 120 contacting with the first insertion hole 110. According to the present embodiment, the bending angle theta is set to approximately 110 degrees. If the bending angle theta lies within a range of from 90 degrees to 150 degrees, since the third tube 38 can be taken out from the second blocking portion 124 without being bent, it is possible for the third tube 38 to be smoothly detached.

The tube detaching portion 126 is formed by a pair of detachment edge portions 126e having the same width as the width between the pair of second straight edge portions 124e, and extends to the outer peripheral frame 106 of the main body 102. The pair of detachment edge portions 126e form, on the long side 106d, a detachment opening 127 that allows the second insertion hole 120 to communicate with the outer side of the main body 102. The width between the pair of detachment edge portions 126e is set to be approximately the same as the width between the pair of second straight edge portions 124e (or slightly wider than the width between the pair of second straight edge portions 124e).

Further, the clamp 100 includes a pair of second protrusions 128 on the second blocking portion 124 that project toward an inner side of the second insertion hole 120. The pair of second protrusions 128 are disposed at positions in the vicinity of the second opening portion 122. Moreover, the pair of second protrusions 128 may be provided at a boundary between the second opening portion 122 and the second blocking portion 124.

The pair of second protrusions 128 are connected respectively to one and the other of the pair of second straight edge portions 124e. The pair of second protrusions 128 project in directions to approach toward each other, thereby narrowing the width of the second blocking portion 124. Moreover, a second protrusion 128 may be provided on only one of the pair of second straight edge portions 124e. Each of the second protrusions 128, in the same manner as the first protrusions 118, is formed in a substantially right triangular shape having an inclined side 128a and a perpendicular side 128b. In addition, the inclined sides 128a are provided so as to face the side of the second opening portion 122, and the perpendicular sides 128b are provided so as to face the arcuate curved portions 124c. It should be noted that the shapes of each of the first protrusions 118 and each of the second protrusions 128 are not limited to being a substantially right triangular shape, and various other shapes such as a wedge shape, a quadrangular shape, and a barb shape or the like may be adopted.

Furthermore, the clamp 100 comprises a pair of detachment side protrusions 130 in the tube detaching portion 126 that project toward an inner side of the second insertion hole 120. The pair of detachment side protrusions 130 are disposed at positions in the vicinity of the second blocking portion 124. Moreover, the pair of detachment side protrusions 130 may be provided at a boundary between the second blocking portion 124 and the tube detaching portion 126.

The pair of detachment side protrusions 130 are connected respectively to one and the other of the pair of detachment edge portions 126e, and each exhibit an equilateral triangular shape having a top rounded corner portion. The pair of detachment side protrusions 130 project in directions to approach toward each other, thereby narrowing the width of the tube detaching portion 126. Consequently, the pair of detachment side protrusions 130 prevent unintentional movement of the third tube 38 between the second blocking portion 124 and the tube detaching portion 126. Moreover, a second protrusion 128 may be provided on only one of the pair of second straight edge portions 124e.

The blood bag system 10 that is configured in the manner described above, as shown in FIG. 3, is provided to a user who is a medical worker, in a state in which the clamp 100 is mounted on the second tube 36 and the third tube 38. Alternatively, the clamp 100 may be provided in a state without being mounted on the tubes 30 and may be mounted on the tubes 30 by the user at the time of being used.

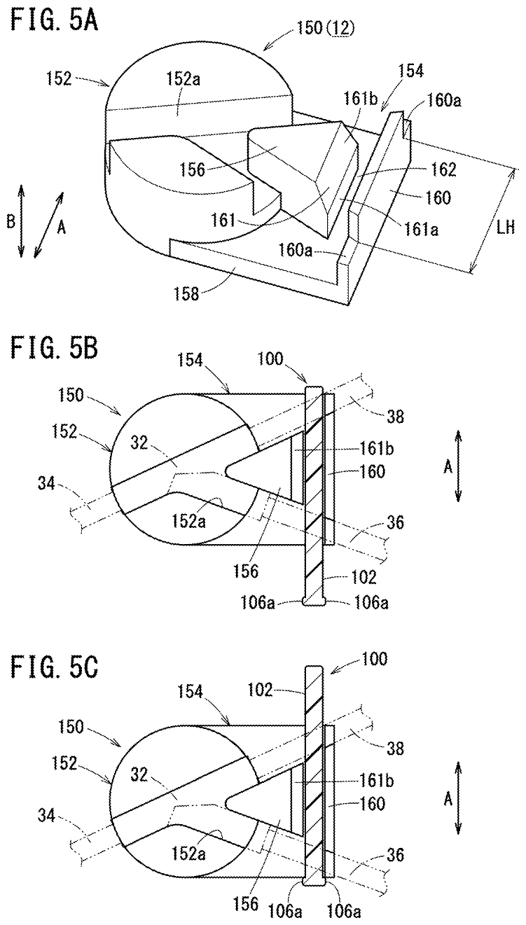

When the blood bag system 10 is set on the centrifugal separation and transferring device 12, the clamp 100 is fixed to the holder 150 together with the branching connector 32. For this purpose, as shown in FIGS. 3 and 5A, the holder 150 includes a connector retaining member 152 in which the branching connector 32 is retained, and a clamp retaining member 154 in which the clamp 100 is retained in a slidable manner.

The connector retaining member 152 is formed in a cylindrical columnar shape, and includes a Y-shaped retaining groove 152a that is cut out to a predetermined depth from the upper end portion. The direction in which the retaining groove 152a extends coincides with the first to third ports 32a to 32c of the branching connector 32, and further, a groove width of the retaining groove 152a is set to a dimension that enables the first to third ports 32a to 32c to be fitted therein from the exterior. A substantially triangular shaped tube guide 156, which constitutes a branching portion of the retaining groove 152a that extends in a straight line shape, is connected to the connector retaining member 152.

The clamp retaining member 154 is connected to the projecting direction side of the tube guide 156 (the side away from the branching portion of the retaining groove 152a). The clamp retaining member 154 includes a base plate 158 that extends from a lower part of the connector retaining member 152 and beyond the tube guide 156, a retaining plate 160 erected at a projecting end of the base plate 158, and an opposing wall 161 constituted by the tube guide 156 and facing toward the retaining plate 160. The retaining plate 160 and the opposing wall 161 constitute a pair of wall portions for retaining the clamp 100. The clamp retaining member 154 supports the clamp 100 such that the clamp 100 can slide in a horizontal direction (in the direction of the arrow A), by way of an arrangement space 162 that is sandwiched between a pair of wall portions (the retaining plate 160 and the opposing wall 161).

The tube guide 156 gradually widens so as to be continuous with the branching shape of the retaining groove 152a, whereby the second tube 36 and the third tube 38 in close proximity to the branching connector 32 extend in straight line shapes. A lower side 161a of the opposing wall 161 is formed in parallel with the retaining plate 160. The lower side 161a (on the side of the base plate 158) of the opposing wall 161 is erected in a direction perpendicular to the base plate 158, whereas an upper side 161b of the opposing wall 161 is formed as an inclined surface that is inclined with respect to the lower side 161a so as to widen the arrangement space 162. Stated otherwise, the upper side 161b of the opposing wall 161 is inclined upward so as to separate away from the retaining plate 160.

The retaining plate 160 is formed in parallel with the lower side 161a of the opposing wall 161. Consequently, the distance between the lower side 161a of the opposing wall 161 and the retaining plate 160 (the thickness of the arrangement space 162) is constant in the vertical direction. The thickness of the arrangement space 162 substantially coincides with the thickness of the main body 102 of the clamp 100. In a state of being disposed in the arrangement space 162, the clamp 100 is sandwiched with an appropriate frictional force between the tube guide 156 and the retaining plate 160. Further, both ends of the retaining plate 160 in the direction of extension thereof are appropriately cut out to thereby include a pair of steps 160a. The steps 160a support the respective lower sides of the second tube 36 and the third tube 38.

A lateral length LH of the protruding upper portion of the retaining plate 160 that is sandwiched between the pair of steps 160a substantially coincides with the interval, or distance, LO (see, e.g., FIG. 4) between the first opening portion 112 of the first insertion hole 110 and the second opening portion 122 of the second insertion hole 120 of the clamp 100. Accordingly, as shown in FIG. 3, in a state when the blood bag system 10 is set, the second opening portion 122 of the clamp 100 and the retaining plate 160 sandwich the third tube 38 therebetween. Further, the step 160a of the retaining plate 160 on the side on which the second tube 36 is arranged blocks the tube introducing portion 116 in a state when the blood bag system 10 is set. Thus, the second tube 36 is prevented from coming off from the clamp 100.

The blood bag system 10 and the centrifugal separation system 14 according to the first embodiment are basically configured in the manner described above. Next, a description will be given below concerning operations thereof.

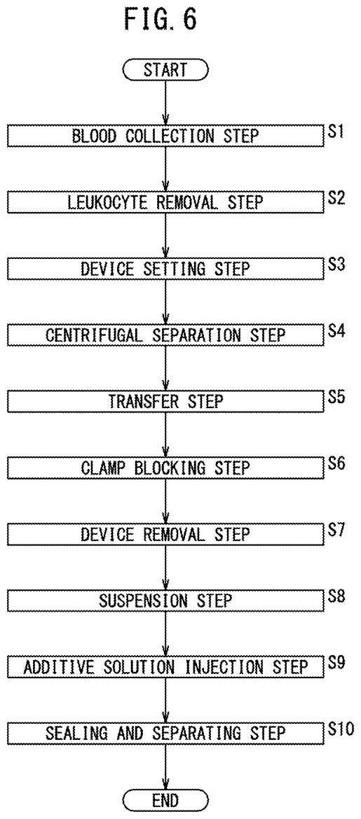

In the blood bag system 10, as shown in FIG. 6, a flow of operations, made up from a blood collection step (step S1), a leukocyte removal step (step S2), a device setting step (step S3), a centrifugal separation step (step S4), a transfer step (step S5), a clamp blocking step (step S6), a device removal step (step S7), a suspension step (step S8), an additive solution injection step (step S9), and a sealing and separating step (step S10), are executed sequentially by the user.

In the blood collection step, the user collects whole blood from a blood donor using a blood collection needle of a non-illustrated preprocessing unit, and stores the collected blood in a blood collection bag. In the leukocyte removal step, the user moves the whole blood from the blood collection bag into the blood bag 22, and at this time, by the whole blood passing through the filter, the removed blood from which leukocytes have been removed from the whole blood is stored in the blood bag 22.

In order for the removed blood to be subjected to centrifugal separation, in the device setting step, the user separates the separation processing unit 16 of the blood bag system 10 away from the preprocessing unit, and sets it in the centrifugal separation and transferring device 12. In the case that the clamp 100 is provided separately from the blood bag system 10, the user mounts the clamp 100 on the second tube 36 and the third tube 38 in the vicinity of the branching connector 32. Upon mounting thereof, the user inserts the second tube 36 from the introduction opening 117 of the clamp 100, and guides the second tube 36 into the first opening portion 112 through the tube introducing portion 116. Furthermore, the user inserts the third tube 38 from the detachment opening 127 of the clamp 100, and guides the third tube 38 into the second opening portion 122 through the tube detaching portion 126 and the second blocking portion 124.

As shown in FIG. 2, the user accommodates the blood bag 22 in the blood bag pocket 56 of the unit setting area 54, and further, sets the first to third tubes 34, 36, and 38 along predetermined paths on the upper surface 54a of the unit setting area 54. The first tube 34 is arranged so as to proceed toward the left region 54a2, after being routed through the predetermined path in the central region 54a1. Further, the user passes the first to third tubes 34, 36, and 38 through the left region 54a2, and retains the branching connector 32 and the clamp 100 in the holder 150. The second tube 36 is arranged so as to extend through the left region 54a2 from the branching connector 32 to the outer side in the direction of centrifugation, and to pass through the PPP clamp 70. Furthermore, the second tube 36 is accommodated in the guide groove portion 79 of the tube retaining member 76 on an outer side in the radial direction of the chemical solution bag pocket 60. The third tube 38 also is arranged so as to extend through the left region 54a2 from the branching connector 32 to the outer side in the direction of centrifugation, and to pass through the chemical solution clamp 72. In addition, the user accommodates, in the PPP bag pocket 58, the PPP bag 24 that is connected to the second tube 36, and accommodates, in the chemical solution bag pocket 60, the chemical solution bag 26 that is connected to the third tube 38. Consequently, setting of the blood bag system 10 on the centrifugal separation and transferring device 12 is completed.

Further, at the time of setting, in a state in which the second tube 36 is arranged in the first opening portion 112 of the clamp 100, and the third tube 38 is arranged in the second opening portion 122 of the clamp 100, the user moves the clamp 100 to a position in the vicinity of the branching connector 32. In addition, as shown in FIG. 3, the user mounts the branching connector 32 in the connector retaining member 152 of the holder 150, while at the same time, inserts the clamp 100 into the arrangement space 162 between the tube guide 156 and the retaining plate 160 of the holder 150.

In the clamp 100, at the time of the device setting step, the second tube 36 is arranged in the first opening portion 112, whereas the third tube 38 is arranged in the second opening portion 122. Accordingly, the second flow path 36a of the second tube 36 and the third flow path 38a of the third tube 38 are opened. Further, due to being supported on the pair of steps 160a, while the protruding upper portion of the retaining plate 160 is arranged therebetween, the second tube 36 and the third tube 38 extend in straight line shapes and in directions to separate away from each other.

Thereafter, in the centrifugal separation step, the centrifugal separation and transferring device 12 causes the centrifugal drum 48 to rotate under the control of the control unit 50, thereby subjecting the removed blood from the blood bag 22 to centrifugal separation into blood components such as PPP, RBC and the like having different specific gravities. During centrifugal separation, the second and third tubes 36 and 38 are blocked by the PPP clamp 70 and the chemical solution clamp 72, and flowing of the blood components therein is prevented.

Further, in the transfer step, the centrifugal separation and transferring device 12 opens only the PPP clamp 70 (see, e.g., FIG. 2) to press the blood bag 22 by the slider 82. Consequently, the PPP from the blood bag 22 flows sequentially through the first tube 34, the branching connector 32, and the second tube 36, and flows into the PPP bag 24.

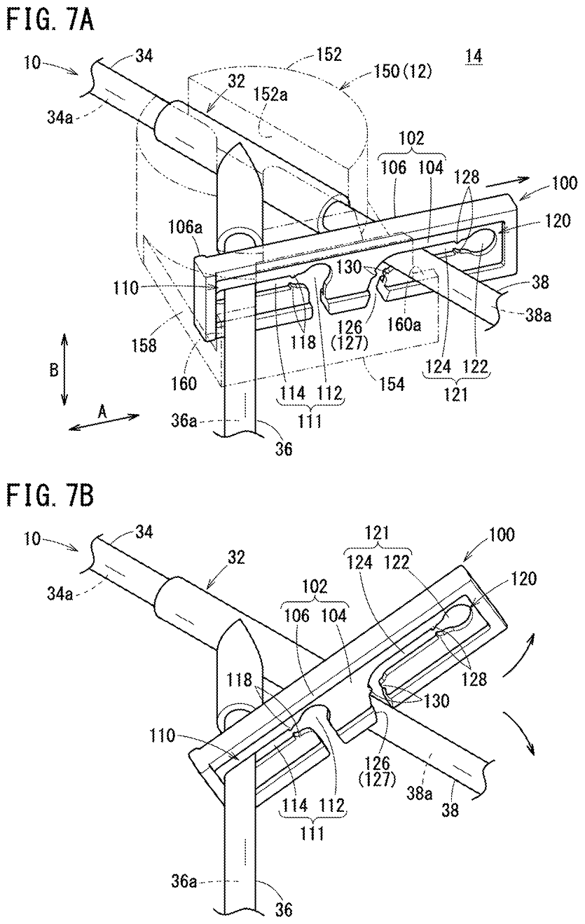

After the transfer step, the user carries out the clamp blocking step to block the second tube 36 and the third tube 38 by the clamp 100. At this time, the user accesses the upper surface 54a of each of the unit setting areas 54, and as shown in FIG. 7A, causes the clamp 100 to slide with respect to the holder 150 toward the other end side in the longitudinal direction. During this sliding, by coming into contact with the retaining plate 160, the second tube 36 does not slide together with the clamp 100. Further, due to being connected to the second tube 36 via the branching connector 32, the third tube 38 is fixed on the step 160a, and does not slide together with the clamp 100.

Thus, during this sliding, the first insertion hole 110 moves relatively with respect to the second tube 36, and the second tube 36 passes beyond the pair of first protrusions 118 and moves into the first blocking portion 114. Consequently, the first blocking portion 114 blocks the second flow path 36a of the second tube 36. Similarly, during the sliding, the second insertion hole 120 moves relatively with respect to the third tube 38, and the third tube 38 passes beyond the pair of second protrusions 128 and moves into the second blocking portion 124. Consequently, the second blocking portion 124 blocks the third flow path 38a of the third tube 38.

Further, as shown in FIGS. 5B and 5C, during the sliding, at a stage at which the flange portions 106a come into contact with the retaining plate 160 of the holder 150, the clamp 100 is restricted from sliding beyond that point. At this stage, the second tube 36 is reliably moved into the first blocking portion 114, and the third tube 38 is reliably moved into the second blocking portion 124.

Next, in the device removal step, in the centrifugal separation and transferring device 12, by retracting the slider 82 and opening the chemical solution clamp 72 (see, e.g., FIG. 2), the blood bag system 10 can be detached from the centrifugal separation and transferring device 12, and the user takes out the blood bag system 10. At this time, due to the upper side 161b of the protruding end portion of the tube guide 156 being an inclined surface, the user is capable of easily accessing the clamp 100 that is retained by the holder 150, and removing the clamp 100. Then, in the suspension step, the user suspends the chemical solution bag 26 on a non-illustrated stand, and furthermore, arranges the blood bag 22 on a lower side in the direction of gravity than the position of the chemical solution bag 26.

Further, in the device removal step and the suspension step, each of the first protrusions 118 prevents the second tube 36 from moving from the first blocking portion 114 into the first opening portion 112, and each of the second protrusions 128 prevents the third tube 38 from moving from the second blocking portion 124 into the second opening portion 122. Furthermore, in the device removal step and the suspension step, each of the detachment side protrusions 130 restricts the third tube 38 from being unintentionally moved from the second blocking portion 124 into the tube detaching portion 126.

In the additive solution injection step, by detaching the third tube 38 from the clamp 100 and breaking the sealing member 42, the user supplies the additive solution in the chemical solution bag 26 to the blood bag 22. Before the third tube 38 is detached, on the clamp 100, the first blocking portion 114 that is blocking the second tube 36 is closed by the main body 102, while on the other hand, the second blocking portion 124 that is blocking the third tube 38 is directly in communication with the tube detaching portion 126. Therefore, as shown in FIG. 7B, the user can avoid misidentifying or mistaking the second tube 36 and the third tube 38, and can easily move the third tube 38 into the tube detaching portion 126 and detach the third tube 38 from the detachment opening 127 of the main body 102. In particular, the tube detaching portion 126 extends in a direction that differs from the direction in which the second blocking portion 124 extends. Therefore, the second tube 36 receives almost no influence of the force applied to the clamp 100 or the third tube 38 when the user detaches the third tube 38. As a result, the clamp 100 enables the third tube 38 to be detached, while suppressing movement of the second tube 36 from the first blocking portion 114 into the first opening portion 112.

In the sealing and separating step after the additive solution injection step, the user seals and severs an appropriate position of the first tube 34. Consequently, the blood bag 22 is separated away from the blood bag system 10 in a state in which the RBC with the additive solution contained therein is stored. Similarly, on the second tube 36, the user seals and severs a portion thereof more on the side of the PPP bag 24 than the clamp 100. Consequently, the PPP bag 24 is separated away from the blood bag system 10 in a state with the PPP stored therein. The remaining parts of the blood bag system 10 are appropriately discarded. At this time, the clamp 100 may be removed from the blood bag system 10, and used again when another blood bag system 10 is placed in use.

The present disclosure is not limited to the above-described embodiment, and various modifications can be adopted in accordance with the essence and gist of the present disclosure. For example, the clamp 100 may have a configuration in which the entirety of the first insertion hole 110 is non-open (e.g., is closed by the main body 102) without the tube introducing portion 116 being provided in the first insertion hole 110. In accordance with this feature, it is possible to prevent the second tube 36 from becoming detached from the clamp 100. In this case, during manufacturing of the blood bag system 10, after the main body 102 having the first insertion hole 110 and the second insertion hole 120 has been molded, initially, the second tube 36 may be passed through the first insertion hole 110, and thereafter, fixing of the second tube 36 and the branching connector 32 may be carried out.

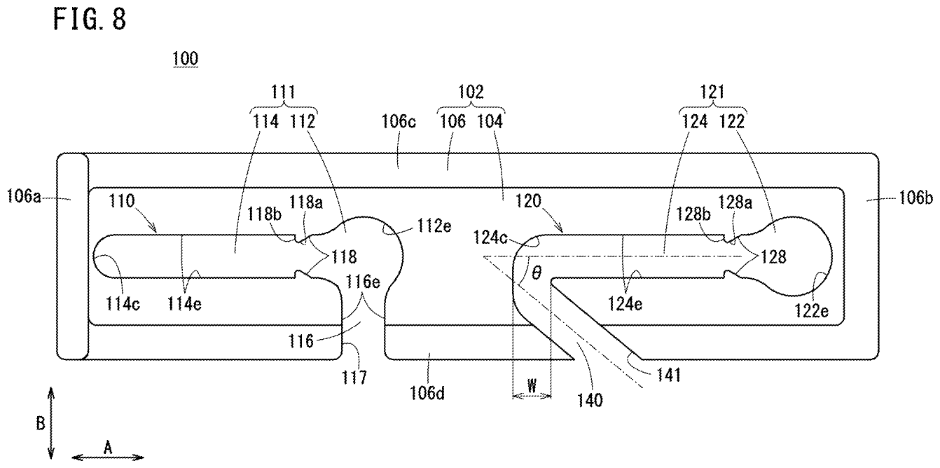

Further, a second insertion hole 120 having a tube detaching portion 140 as shown in FIG. 8 may be formed in the main body 102 of the clamp 100. More specifically, the tube detaching portion 140 extends in a direction of being folded back (a direction forming an acute angle: a bending angle theta of 20 degrees to 89 degrees) with respect to the direction in which the second blocking portion 124 extends from the second opening portion 122. The tube detaching portion 140 has a detachment opening 141 on a lower side of the second blocking portion 124, and a width W of the folded back point is formed to be narrower than the width of the pair of second straight edge portions 124e. The tube detaching portion 140 formed in this manner does not allow the third tube 38, that has been moved into the second blocking portion 124, to be easily moved into the tube detaching portion 140, and effectively prevents the third tube 38 from being unintentionally detached.

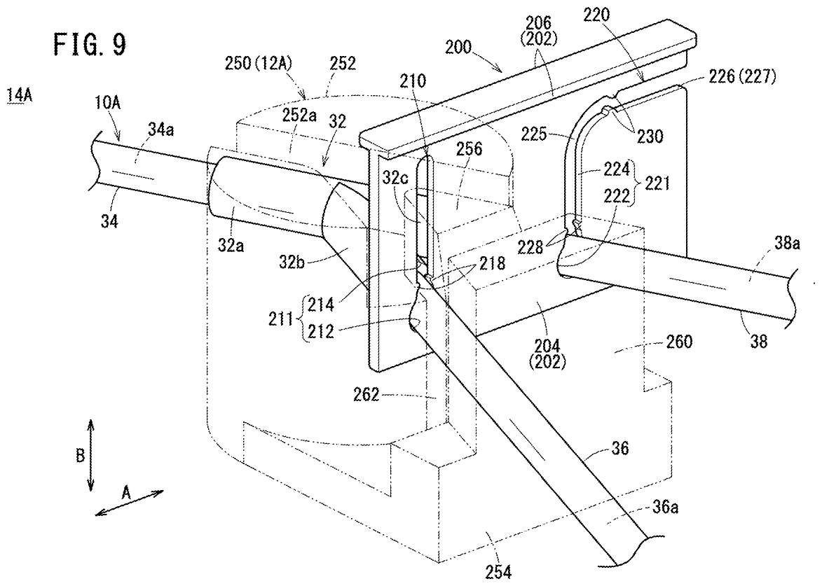

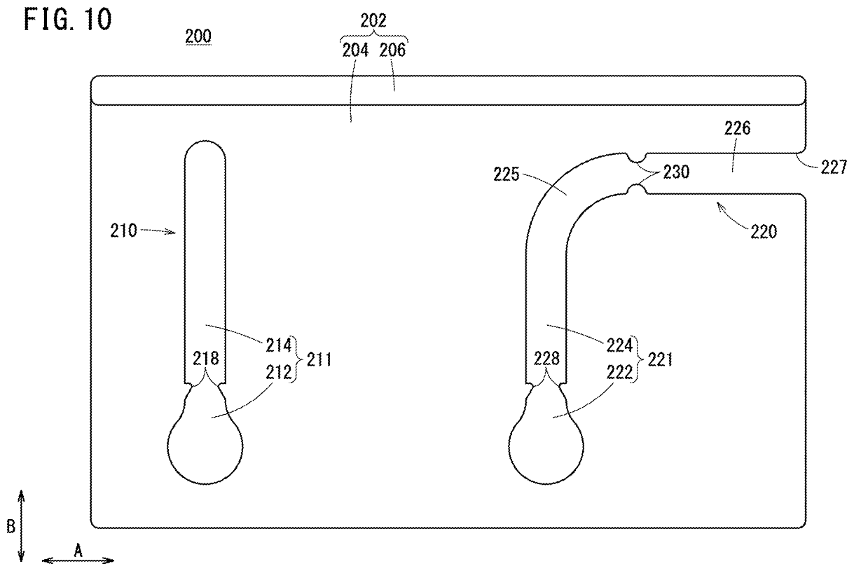

Next, with reference to FIGS. 9 to 11B, a description is provided concerning a blood bag system 10A according to a second embodiment of the present disclosure. A clamp 200 of the blood bag system 10A differs from the clamp 100 of the blood bag system 10, in that a direction of extension of a first space 211 of a first insertion hole 210 in which a first opening portion 212 and a first blocking portion 214 are disposed alongside one another, and a direction of extension of a second space 221 of a second insertion hole 220 in which a second opening portion 222 and a second blocking portion 224 are disposed alongside one another, extend along a lateral direction of a main body 202. In the following description, constituent elements (constituent elements apart from the clamp 200) having the same configuration or the same function as those of the above-described embodiment are designated by the same reference numerals, and detailed description of such features is omitted.

More specifically, the main body 202 of the clamp 200 includes a base 204 having a horizontally elongate rectangular shape, and a pair of flange portions 206 that project from the base 204 on one side in the lateral direction of the base 204, and is formed in a rectangular shape as viewed from the front. The base 204 and the flange portions 206 are integrally molded from the same material.

In addition, the first insertion hole 210 and the second insertion hole 220 are formed by inner edge portions included on an inner side of the base 204. The first space 211 of the first insertion hole 210 and the second space 221 of the second insertion hole 220 are arranged alongside one another in the longitudinal direction of the base 204, and extend in parallel with each other.

The first space 211 includes the first opening portion 212 and the first blocking portion 214, and further, is formed with a configuration in which the entirety thereof is non-open (e.g., is closed by the main body 202). Moreover, the first insertion hole 210 may be configured to include the tube introducing portion 116, in the same manner as the first insertion hole 110 of the first embodiment.

The first opening portion 212 is formed in a substantially perfect circular shape, and the second flow path 36a of the second tube 36 is arranged therein in an open state. The first opening portion 212 is provided in the main body 202 close to a long side that is on the opposite side to the flange portions 206.

The first blocking portion 214 blocks the second flow path 36a by pressing inwardly on the outer circumferential surface of the second tube 36. The first blocking portion 214 is formed as a vertical elongate space that extends in a straight line shape from the first opening portion 212 toward the side of the flange portions 206 of the main body 202. Further, the first blocking portion 214 includes a pair of first protrusions 218 at positions in the vicinity of the first opening portion 212.

The second space 221 includes the second opening portion 222 and the second blocking portion 224. Furthermore, the second insertion hole 220 includes a tube detaching portion 226 that is connected with the second blocking portion 224 and that enables the second tube 36 to be detached from the main body 202.

The second opening portion 222 is formed in a substantially perfect circular shape, and the third flow path 38a of the third tube 38 is arranged therein in an open state. The second opening portion 222 is provided in the main body 202 at the same position in the lateral direction as that of the first opening portion 212 (close to the long side that is on the opposite side to the flange portions 206).

The second blocking portion 224 blocks the third flow path 38a by pressing inwardly on the outer circumferential surface of the third tube 38. The second blocking portion 224 is formed as a vertical elongate space that extends in a straight line shape from the second opening portion 222 toward the side of the flange portions 206 of the main body 202. The side of the second blocking portion 224 closer to the flange portions 206 is formed as a curved passage 225 that is smoothly continuous with the tube detaching portion 226. Further, the second blocking portion 224 includes a pair of second protrusions 228 at positions in the vicinity of the second opening portion 222.

The tube detaching portion 226 extends in a longitudinal direction of the main body 202, that is perpendicular to the direction in which the second blocking portion 224 extends. More specifically, in the second embodiment, the bending angle theta of the direction of extension of the tube detaching portion 226 with respect to the direction of extension of the second blocking portion 224 is set to 90 degrees.

The tube detaching portion 226 is formed with the same width as the width of the second blocking portion 224, and extends to a predetermined short side of the base 204 (a side closer to the second space 221). The tube detaching portion 226 includes, on the short side, a detachment opening 227 that allows the second insertion hole 220 to communicate with the exterior of the main body 202. Further, the tube detaching portion 226 includes a pair of detachment side protrusions 230 at positions in the vicinity of the curved passage 225 of the second blocking portion 224.

In order to set the blood bag system 10A having the above-described clamp 200, a centrifugal separation and transferring device 12A also includes a holder 250 corresponding to the clamp 200. A connector retaining member 252 and a clamp retaining member 254 of the holder 250 are formed so as to project in an elongate manner along the vertical direction (the direction of the arrow B) of the unit setting area 54. Further, a retaining groove 252a of the connector retaining member 252 is formed with a shallow depth, whereas an arrangement space 262 formed between a tube guide 256 and a retaining plate 260 of the clamp retaining member 254 is formed to be deeper than the retaining groove 252a.

The blood bag system 10A, the centrifugal separation system 14A, and the clamp 200 according to the second embodiment are configured in the manner described above. During usage thereof, the same flow of operations as those of the centrifugal separation system 14 according to the first embodiment are carried out. However, in the device setting step, in a state in which the branching connector 32 and the clamp 200 are retained by the holder 250, the clamp 200 exhibits a form of protruding in an upward direction with respect to the holder 250 as shown in FIG. 9. Consequently, the centrifugal separation step and the transfer step are carried out in a state in which the second tube 36 is arranged in the first opening portion 212 of the first insertion hole 210, and the third tube 38 is arranged in the second opening portion 222 of the second insertion hole 220.

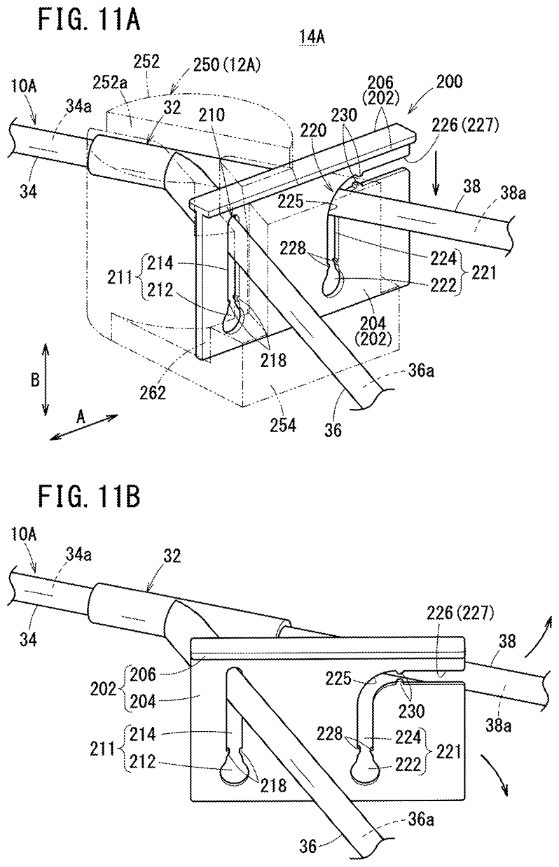

In addition, in the clamp blocking step, as shown in FIG. 11A, the user slides the clamp 200 downward. During this sliding, since the branching connector 32 is retained by the holder 250, the second tube 36 and the third tube 38 do not slide. Therefore, the second tube 36 moves into the first blocking portion 214, and the second flow path 36a is blocked by the first blocking portion 214. Similarly, the third tube 38 moves into the second blocking portion 224, and the third flow path 38a is blocked by the second blocking portion 224.

Furthermore, as shown in FIG. 11B, in the additive solution injection step, the user detaches the third tube 38 from the clamp 200. The direction in which the tube detaching portion 226 extends (the direction of the force applied to the clamp 200 or the third tube 38 by the user during detaching) differs from the direction in which the first insertion hole 210 extends. Therefore, the user is capable of smoothly detaching the third tube 38 from the clamp 200.

Moreover, it is also possible for the clamp 200 to be modified in various ways. For example, the direction in which the first opening portion 212 and the first blocking portion 214 are arranged alongside one another, and the direction in which the second opening portion 222 and the second blocking portion 224 are arranged alongside one another, may be a direction from the flange portions 206 toward the long side opposite thereto. In this case, the tube detaching portion 226 may be appropriately provided so as to be continuous with an extending end portion of the second blocking portion 224 (an end portion thereof on the opposite side to the second opening portion 222).

Technical concepts, advantages, benefits, and effects provided by the above-described embodiments will be described below.

The first aspect of the present disclosure is characterized by the blood bag system 10 or 10A, comprising the first bag (e.g., the blood bag 22) in which blood is accommodated, the second bag (e.g., the PPP bag 24) in which a blood component obtained by subjecting the blood inside the first bag to centrifugal separation is accommodated, the third bag (e.g., the chemical solution bag 26) in which the additive solution is accommodated, the first tube 34 connected to the first bag, the second tube 36 connected to the second bag, and the third tube 38 connected to the third bag, the first tube 34, the second tube 36, and the third tube 38 being connected via a branching portion (e.g., the branching connector 32), the blood bag system further comprising the single clamp 100 or 200 that is mounted over the second tube 36 and the third tube 38, wherein the clamp 100 or 200 includes the main body 102 or 202, the first insertion hole 110 or 210 that penetrates, or passes, through the main body 102 or 202 and in which the second tube 36 is arranged, and the second insertion hole 120 or 220 that penetrates, or passes, through the main body 102 and in which the third tube 38 is arranged, the first insertion hole 110 or 210 has the first opening portion 112 or 212 that places the flow path (e.g., the second flow path 36a) of the second tube 36 in an open state, and the first blocking portion 114 or 214 that is connected with the first opening portion 112 or 212 and that places the flow path of the second tube 36 in a blocked state, and in the first blocking portion 114 or 214, a portion thereof except for a portion connected with the first opening portion 112 or 212 is closed by the main body 102 or 202, and the second insertion hole 120 or 220 has the second opening portion 122 or 222 that places the flow path (e.g., the third flow path 38a) of the third tube 38 in an open state, the second blocking portion 124 or 224 that is connected with the second opening portion 122 or 222 and that places the flow path of the third tube 38 in a blocked state, and the tube detaching portion 126 or 226 that is connected with the second blocking portion 124 or 224 and that enables the third tube 38 to be detached from the main body 102 or 202.

In accordance with the above-described configuration, the blood bag system 10 or 10A is capable of efficiently blocking the flow paths of the two tubes 30 by the single clamp 100 or 200, and preventing the flow path of the tube 30 (e.g., the third tube 38) from being opened by mistake. More specifically, in the first insertion hole 110 or 210 of the clamp 100 or 200, the second tube 36 is not detached from the first blocking portion 114 or 214. On the other hand, the second insertion hole 120 or 220 allows the third tube 38 to be easily detached from the tube detaching portion 126 or 226 that is connected with the second blocking portion 124 or 224. Accordingly, the user can avoid misidentifying or mistaking the tubes 30 whose flow paths should be opened. As a result, the blood bag system 10 or 10A can reduce mishandling of the system after centrifugal separation has been completed, and enables blood products to be accurately obtained.

The first blocking portion 114 or 214 and the second blocking portion 124 or 224, respectively, extend from the first opening portion 112 or 212 and the second opening portion 122 or 222 mutually in the same direction, and the tube detaching portion 126 or 226 extends in a direction that differs from the direction in which the second blocking portion 124 or 224 extends. In accordance with these features, in an operation by the user of detaching the third tube 38 from the clamp 100 or 200, the clamp 100 or 200 is capable of preventing the second tube 36 from being moved into the first opening portion 112 or 212.

Further, the direction in which the tube detaching portion 126 or 226 extends is bent at an angle of 20 degrees to 150 degrees with respect to the direction in which the second blocking portion 124 or 224 extends. In accordance with this feature, while preventing the tubes 30 from being misidentified or mistaken, the user is capable of smoothly moving the third tube 38 from the second blocking portion 124 or 224 into the tube detaching portion 126 or 226.

Further, the first opening portion 112 or 212 is formed in a circular shape having a diameter greater than or equal to the outer diameter of the second tube 36, the first blocking portion 114 or 214 is formed as a straight line shaped space that is capable of blocking the flow path (e.g., the second flow path 36a) of the second tube 36 by inwardly pressing on the outer circumferential surface of the second tube 36, the second opening portion 122 or 222 is formed in a circular shape having a diameter greater than or equal to the outer diameter of the third tube 38, and the second blocking portion 124 or 224 is formed as a straight line shaped space that is capable of blocking the flow path (e.g., the third flow path 38a) of the third tube 38 by inwardly pressing on the outer circumferential surface of the third tube 38. In accordance with these features, the clamp 100 or 200 is capable of more easily carrying out movement of the second tube 36 from the first opening portion 112 or 212 into the first blocking portion 114 or 214, and movement of the third tube 38 from the second opening portion 122 or 222 into the second blocking portion 124 or 224.

Further, the tube detaching portion 126 or 226 is formed to have the same width as the width of the second blocking portion 124 or 224. In accordance with this feature, the user of the clamp 100 or 200 is capable of easily handling the movement of the third tube 38 from the second blocking portion 124 or 224 into the tube detaching portion 126 or 226.

Further, the main body 102 is formed in a rectangular plate-like shape when viewed from the front, the first insertion hole 110 and the second insertion hole 120 are arranged alongside one another in a longitudinal direction of the main body 102, and the direction in which the first space 111 that is made up from the first opening portion 112 and the first blocking portion 114 extends, and the direction in which the second space 121 that is made up from the second opening portion 122 and the second blocking portion 124 extends, are along the longitudinal direction of the main body 102. In accordance with these features, by the user sliding the clamp 100 along the longitudinal direction of the main body 102, movement of the second tube 36 from the first opening portion 112 into the first blocking portion 114, and movement of the third tube 38 from the second opening portion 122 into the second blocking portion 124 can be performed simultaneously.

Further, the main body 202 is formed in a plate-like shape, the first insertion hole 210 and the second insertion hole 220 are arranged alongside one another in a first direction perpendicular to a thickness direction of the main body 202, and the direction in which the first space 211 that is made up from the first opening portion 212 and the first blocking portion 214 extends, and the direction in which the second space 221 that is made up from the second opening portion 222 and the second blocking portion 224 extends, are along a second direction perpendicular to the thickness direction and to the first direction. In accordance with these features, by the user sliding the clamp 200 along the second direction of the main body 202, movement of the second tube 36 from the first opening portion 212 into the first blocking portion 214, and movement of the third tube 38 from the second opening portion 222 into the second blocking portion 224 can be performed simultaneously.

Further, a first protrusion 118 or 218, that projects toward the inner side of the first insertion hole 110 or 210 and prevents movement of the second tube 36 from the first blocking portion 114 or 214 into the first opening portion 112 or 212, is provided on the first blocking portion 114 or 214 or at a boundary portion between the first opening portion 112 or 212 and the first blocking portion 114 or 214, and the second protrusion 128 or 228, that projects toward the inner side of the second insertion hole 120 or 220 and prevents movement of the third tube 38 from the second blocking portion 124 or 224 into the second opening portion 122 or 222, is provided on the second blocking portion 124 or 224 or at a boundary portion between the second opening portion 122 or 222 and the second blocking portion 124 or 224. In accordance with these features, the clamp 100 or 200 is capable of preventing unintentional movement of the second tube 36 from the first blocking portion 114 or 214 into the first opening portion 112 or 212, and unintentional movement of the third tube 38 from the second blocking portion 124 or 224 into the second opening portion 122 or 222.

Further, the detachment side protrusion 130 or 230, that projects toward the inner side of the second insertion hole 120 or 220, is provided on the tube detaching portion 126 or 226 or at a boundary portion between the second blocking portion 124 or 224 and the tube detaching portion 126 or 226. In accordance with this feature, the clamp 100 or 200 is capable of preventing unintentional movement of the third tube 38 from the second blocking portion 124 or 224 into the tube detaching portion 126 or 226.

Further, the first insertion hole 110 includes the tube introducing portion 116 that is connected with the first opening portion 112 and is capable of introducing the second tube 36 into the first opening portion 112. In accordance with this feature, the blood bag system 10 enables the clamp 100 to be attached later, even when the second tube 36, the third tube 38, and the branching connector 32 are connected to each other.

Further, the second aspect of the present disclosure is characterized by the centrifugal separation system 14 or 14A, comprising: the blood bag system 10 or 10A including the first bag (e.g., the blood bag 22) in which blood is accommodated, the second bag (e.g., the PPP bag 24) in which a blood component obtained by subjecting the blood inside the first bag to centrifugal separation is accommodated, the third bag (e.g., the chemical solution bag 26) in which the additive solution is accommodated, the first tube 34 connected to the first bag, the second tube 36 connected to the second bag, and the third tube 38 connected to the third bag, the first tube 34, the second tube 36, and the third tube 38 being connected via a branching portion (e.g., the branching connector 32); and the centrifugal separation and transferring device 12 or 12A in which the blood bag system 10 or 10A is set, and that subjects the blood in the first bag to centrifugal separation, and transfers the blood component into the second bag, wherein the blood bag system 10 or 10A includes the single clamp 100 or 200 that is mounted over the second tube 36 and the third tube 38, the clamp 100 or 200 includes the main body 102 or 202, the first insertion hole 110 or 210 that penetrates, or passes, through the main body 102 or 202 and in which the second tube 36 is arranged, and the second insertion hole 120 or 220 that penetrates through the main body 102 or 202 and in which the third tube 38 is arranged, the first insertion hole 110 or 210 has the first opening portion 112 or 212 that places the flow path (e.g., the second flow path 36a) of the second tube 36 in an open state, and the first blocking portion 114 or 214 that is connected with the first opening portion 112 or 212 and that places the flow path of the second tube 36 in a blocked state, and in the first blocking portion 114 or 214, a portion thereof except for a portion connected with the first opening portion 112 or 212 is closed by the main body 102 or 202, the second insertion hole 120 or 220 has the second opening portion 122 or 222 that places the flow path (e.g., the third flow path 38a) of the third tube 38 in an open state, the second blocking portion 124 or 224 that is connected with the second opening portion 122 or 222 and that places the flow path of the third tube 38 in a blocked state, and the tube detaching portion 126, 140, or 226 that is connected with the second blocking portion 124 or 224 and that enables the third tube 38 to be detached from the main body 102 or 202, and the centrifugal separation and transferring device 12 includes the holder 150 or 250 in which the branching portion and the clamp 100 or 200 are retained. In accordance with such a configuration, the centrifugal separation system 14 or 14A is capable of efficiently blocking the flow paths of the two tubes 30 by the single clamp 100 or 200 that is arranged in the holder 150 or 250, and preventing the flow path of the tube 30 (e.g., the third tube 38) from being opened by mistake.

Further, the holder 150 or 250 comprises the branching portion retaining member (e.g., the connector retaining member 152 or 252) in which the branching portion (e.g., the branching connector 32) is retained, and the clamp retaining member 154 or 254 that sandwiches and retains the clamp 100 or 200 in a slidable manner at a position adjacent to the branching portion retaining member. In accordance with these features, the centrifugal separation system 14 or 14A can stably subject the clamp 100 or 200 to a sliding operation at a position adjacent to the branching portion.

Further, the clamp retaining member 154 or 254 comprises the pair of wall portions (e.g., the retaining plate 160 and the opposing wall 161) sandwiching the clamp 100 or 200 therebetween, and on an upper part of one of the wall portions (e.g., the opposing wall 161) from among the pair of wall portions, the inclined surface (161b), that is inclined upwardly and in a direction away from the other wall portion (e.g., the retaining plate 160), is provided. In accordance with these features, the user is capable of easily taking out the clamp 100 or 200 from the arrangement space 162 or 262.

Further, the main body 102 is formed in a rectangular plate-like shape when viewed from the front, the first insertion hole 110 and the second insertion hole 120 are arranged alongside one another in a longitudinal direction of the main body 102, the direction in which the first space 111 which is made up from the first opening portion 112 and the first blocking portion 114 extends, and the direction in which the second space 121 that is made up from the second opening portion 122 and the second blocking portion 124 extends, are along the longitudinal direction of the main body 102, the clamp retaining member 154 supports the clamp 100 in a slidable manner along a horizontal direction, and when the clamp 100 is made to slide in the horizontal direction from the first position to the second position with respect to the clamp retaining member 154, the clamp 100 blocks the second tube 36 and the third tube 38, respectively, by the first blocking portion 114 and the second blocking portion 124. In accordance with these features, the user can smoothly block the second tube 36 and the third tube 38 by subjecting the clamp 100 to the sliding operation in the horizontal direction.

Further, the main body 202 is formed in a plate-like shape, the first insertion hole 210 and the second insertion hole 220 are arranged alongside one another in a first direction perpendicular to a thickness direction of the main body 202, the direction in which the first space 211 that is made up from the first opening portion 212 and the first blocking portion 214 extends, and the direction in which the second space 221 that is made up from the second opening portion 222 and the second blocking portion 224 extends, are along a second direction perpendicular to the thickness direction and to the first direction, the clamp retaining member 254 supports the clamp 200 in a slidable manner along a vertical direction, in a state in which the second direction is oriented in the vertical direction, and when the clamp 200 is made to slide from a first height to a second height with respect to the clamp retaining member 254, the clamp 200 blocks the second tube 36 and the third tube 38, respectively, by the first blocking portion 214 and the second blocking portion 224. In accordance with these features, the user can smoothly block the second tube 36 and the third tube 38 by subjecting the clamp 200 to the sliding operation in an upward direction or a downward direction.

Further, the third aspect of the present disclosure is characterized by the clamp 100 or 200 that is mounted over the two tubes 30, the clamp comprising the main body 102 or 202, the first insertion hole 110 or 210 that penetrates, or passes, through the main body 102 or 202 and in which one tube 30 of the two tubes 30 is arranged, and the second insertion hole 120 or 220 that penetrates, or passes, through the main body 102 or 202 and in which another tube 30 of the two tubes 30 is arranged, wherein the first insertion hole 110 or 210 has the first opening portion 112 or 212 that places the one tube 30 in an open state, and the first blocking portion 114 or 214 that is connected with the first opening portion 112 or 212 and that places the flow path of the one tube 30 in a blocked state, and in the first blocking portion 114 or 214, a portion thereof except for a portion connected with the first opening portion 112 or 212 is closed by the main body 102 or 202, and the second insertion hole 120 or 220 has the second opening portion 122 or 222 that places the flow path of the other tube 30 in an open state, the second blocking portion 124 or 224 that is connected with the second opening portion 122 or 222 and that places the flow path of the other tube 30 in a blocked state, and the tube detaching portion 126, 140, or 226 that is connected with the second blocking portion 124 or 224 and that enables the other tube 30 to be detached from the main body 102 or 202. In accordance with these features, the clamp 100 or 200 is capable of efficiently blocking the flow paths of the two tubes 30, and preventing the flow path of the tube 30 from being opened by mistake.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

It is to be appreciated that any feature described herein can be claimed in combination with any other feature(s) as described herein, regardless of whether the features come from the same described embodiment.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or a class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The terms “determine,” “calculate,” “compute,” and variations thereof, as used herein, are used interchangeably and include any type of methodology, process, mathematical operation, or technique.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. A blood bag system, comprising:
a first bag configured to accommodate blood;
a second bag configured to accommodate a blood component obtained by subjecting the blood inside the first bag to centrifugal separation;
a third bag configured to accommodate an additive solution;
a first tube connected to the first bag;
a second tube connected to the second bag; and
a third tube connected to the third bag,
the first tube, the second tube, and the third tube being connected via a branching portion,
the blood bag system further comprising a single clamp mounted over the second tube and the third tube, wherein the clamp includes:
a main body;
a first insertion hole that penetrates through the main body and in which the second tube is arranged; and
a second insertion hole that penetrates through the main body and in which the third tube is arranged,
the first insertion hole includes a first opening portion configured to place a flow path of the second tube in an open state, and a first blocking portion connected with the first opening portion and configured to place the flow path of the second tube in a blocked state, and in the first blocking portion, a portion thereof except for a portion connected with the first opening portion is closed by the main body, and
the second insertion hole includes a second opening portion configured to place a flow path of the third tube in an open state, a second blocking portion connected with the second opening portion and configured to place the flow path of the third tube in a blocked state, and a tube detaching portion connected with the second blocking portion and configured to enable the third tube to be detached from the main body.

2. The blood bag system according to claim 1, wherein:
the first blocking portion and the second blocking portion respectively extend from the first opening portion and the second opening portion mutually in a same direction; and
the tube detaching portion extends in a direction that differs from a direction in which the second blocking portion extends.

3. The blood bag system according to claim 2, wherein the direction in which the tube detaching portion extends is bent at an angle of 20 degrees to 150 degrees with respect to the direction in which the second blocking portion extends.

4. The blood bag system according to claim 1, wherein:
the first opening portion is formed in a circular shape having a diameter greater than or equal to an outer diameter of the second tube;
the first blocking portion is formed as a straight line shaped space configured to block the flow path of the second tube by inwardly pressing on an outer circumferential surface of the second tube;
the second opening portion is formed in a circular shape having a diameter greater than or equal to an outer diameter of the third tube; and
the second blocking portion is formed as a straight line shaped space configured to block the flow path of the third tube by inwardly pressing on an outer circumferential surface of the third tube.

5. The blood bag system according to claim 4, wherein the tube detaching portion is formed to have a same width as a width of the second blocking portion.

6. The blood bag system according to claim 1, wherein:
the main body is formed in a rectangular plate-like shape when viewed from a front side;
the first insertion hole and the second insertion hole are arranged alongside one another in a longitudinal direction of the main body; and
a direction in which a first space made up from the first opening portion and the first blocking portion extends, and a direction in which a second space made up from the second opening portion and the second blocking portion extends, are along the longitudinal direction of the main body.

7. The blood bag system according to claim 1, wherein:
the main body is formed in a plate-like shape;
the first insertion hole and the second insertion hole are arranged alongside one another in a first direction perpendicular to a thickness direction of the main body; and
a direction in which a first space made up from the first opening portion and the first blocking portion extends, and a direction in which a second space made up from the second opening portion and the second blocking portion extends, are along a second direction perpendicular to the thickness direction and to the first direction.

8. The blood bag system according to claim 1, wherein:
a first protrusion projecting toward an inner side of the first insertion hole and configured to prevent movement of the second tube from the first blocking portion into the first opening portion is provided on the first blocking portion or at a boundary portion between the first opening portion and the first blocking portion; and
a second protrusion projecting toward an inner side of the second insertion hole and configured to prevent movement of the third tube from the second blocking portion into the second opening portion is provided on the second blocking portion or at a boundary portion between the second opening portion and the second blocking portion.

9. The blood bag system according to claim 1, wherein a detachment side protrusion projecting toward an inner side of the second insertion hole is provided on the tube detaching portion or at a boundary portion between the second blocking portion and the tube detaching portion.

10. The blood bag system according to claim 1, wherein the first insertion hole includes a tube introducing portion connected with the first opening portion and configured to introduce the second tube into the first opening portion.

11. A centrifugal separation system, comprising:
a blood bag system including
a first bag configured to accommodate blood,
a second bag configured to accommodate a blood component obtained by subjecting the blood inside the first bag to centrifugal separation,
a third bag configured to accommodate an additive solution,
a first tube connected to the first bag,
a second tube connected to the second bag, and
a third tube connected to the third bag,
the first tube, the second tube, and the third tube being connected via a branching portion; and
a centrifugal separation and transferring device in which the blood bag system is set, the centrifugal separation and transferring device being configured to subject the blood in the first bag to centrifugal separation and transfer the blood component into the second bag, wherein
the blood bag system includes a single clamp mounted over the second tube and the third tube,
the clamp includes:
a main body;
a first insertion hole that penetrates through the main body and in which the second tube is arranged; and
a second insertion hole that penetrates through the main body and in which the third tube is arranged,
the first insertion hole includes a first opening portion configured to place a flow path of the second tube in an open state, and a first blocking portion connected with the first opening portion and configured to place the flow path of the second tube in a blocked state, and in the first blocking portion, a portion thereof except for a portion connected with the first opening portion is closed by the main body,
the second insertion hole includes a second opening portion configured to place a flow path of the third tube in an open state, a second blocking portion connected with the second opening portion and configured to place the flow path of the third tube in a blocked state, and a tube detaching portion connected with the second blocking portion and configured to enable the third tube to be detached from the main body, and
the centrifugal separation and transferring device includes a holder configured to retain the branching portion and the clamp.

12. The centrifugal separation system according to claim 11, wherein the holder includes a branching portion retaining member configured to retain the branching portion, and a clamp retaining member configured to sandwich and retain the clamp in a slidable manner at a position adjacent to the branching portion retaining member.

13. The centrifugal separation system according to claim 12, wherein:
the clamp retaining member includes a pair of wall portions sandwiching the clamp therebetween; and
on an upper part of one of the pair of wall portions, an inclined surface inclined upwardly and in a direction away from another of the pair of wall portions is provided.

14. The centrifugal separation system according to claim 12, wherein:
the main body is formed in a rectangular plate-like shape when viewed from a front side;
the first insertion hole and the second insertion hole are arranged alongside one another in a longitudinal direction of the main body;
a direction in which a first space made up from the first opening portion and the first blocking portion extends, and a direction in which a second space made up from the second opening portion and the second blocking portion extends, are along the longitudinal direction of the main body;
the clamp retaining member supports the clamp in a slidable manner along a horizontal direction; and
when the clamp is made to slide in the horizontal direction from a first position to a second position with respect to the clamp retaining member, the clamp blocks the second tube and the third tube, respectively, by the first blocking portion and the second blocking portion.

15. The centrifugal separation system according to claim 12, wherein:
the main body is formed in a plate-like shape;
the first insertion hole and the second insertion hole are arranged alongside one another in a first direction perpendicular to a thickness direction of the main body;
a direction in which a first space made up from the first opening portion and the first blocking portion extends, and a direction in which a second space made up from the second opening portion and the second blocking portion extends, are along a second direction perpendicular to the thickness direction and to the first direction;
the clamp retaining member supports the clamp in a slidable manner along a vertical direction, in a state in which the second direction is oriented in the vertical direction; and
when the clamp is made to slide from a first height to a second height with respect to the clamp retaining member, the clamp blocks the second tube and the third tube, respectively, by the first blocking portion and the second blocking portion.

16. A clamp mounted over two tubes, the clamp comprising:
a main body;
a first insertion hole that penetrates through the main body and in which one tube of the two tubes is arranged; and
a second insertion hole that penetrates through the main body and in which another tube of the two tubes is arranged, wherein
the first insertion hole includes a first opening portion configured to place a flow path of the one tube in an open state, and a first blocking portion connected with the first opening portion and configured to place the flow path of the one tube in a blocked state, and in the first blocking portion, a portion thereof except for a portion connected with the first opening portion is closed by the main body, and
the second insertion hole includes a second opening portion configured to place a flow path of the another tube in an open state, a second blocking portion connected with the second opening portion and configured to place the flow path of the another tube in a blocked state, and a tube detaching portion connected with the second blocking portion and configured to enable the another tube to be detached from the main body.

17. The clamp according to claim 16, wherein the first opening portion is arranged an interval distance from the second opening portion, and wherein the first opening portion is separated from the second opening portion by a solid portion of the main body.

18. The clamp according to claim 17, wherein the first opening portion comprises an aperture having a first opening diameter, and wherein the first blocking portion comprises a slot extending a slot length from the first opening diameter to a point inside an outer periphery of the main body.

19. The clamp according to claim 18, wherein a slot width is defined between opposing sidewalls of the slot, and wherein the slot width is less than the first opening diameter.

20. The clamp according to claim 19, wherein a pair of protrusions are arranged along the slot length adjacent the first opening portion, wherein the pair of protrusions comprises a first protrusion extending from a first sidewall of the opposing sidewalls into a first portion of the slot, wherein the pair of protrusions comprises a second protrusion extending from a second sidewall of the opposing sidewalls into a second portion of slot, wherein the first sidewall is arranged opposite the second sidewall, and wherein the first protrusion is separated from the second protrusion by a separation distance.
